(19) United States
(12) Patent Application Publication
(10) Pub. No.: US 2005/0267564 A1
(43) Pub. Date: Dec. 1, 2005

(54) Capsulated stent and its uses

(75) Inventors: Lilong Tang, Windsor (CA); Julius M. Gardin, West Bloomfield, MI (US); Ruth Moore, Huntington Woods, MI (US)

Correspondence Address: JENKENS & GILCHRIST, P.C., 225 WEST WASHINGTON, SUITE 2600, CHICAGO, IL, 60606, US

(73) Assignee: St. John Health

(21) Appl. No.: 11/139,718

(22) Filed: May 27, 2005

Related U.S. Application Data
(60) Provisional application No. 60/574,854, filed on May 27, 2004.

(51) Int. Cl. A61F 2/06

(52) U.S. Cl. 623/1.13; 623/1.42

(57) ABSTRACT

A medical device is provided which includes an implantable medical device that is at least substantially covered in a granulation tissue. The granulation tissue is substantially immunocompatible with an immune system of a patient into which the implantable medical device is to be implanted for a therapeutic purpose.

### CROSS-REFERENCE TO RELATED APPLICATIONS

The present invention claims priority from U.S. Provisional Application Ser. No. 60/574,854, filed May 27, 2004, incorporated herein by reference in its entirety.

### FIELD OF THE INVENTION

The present invention relates generally to a medical device and procedure for the treatment of vascular diseases, particularly to vascular stenosis, the prevention of in-stent restenosis, vascular aneurysms and thrombosis.

### BACKGROUND OF THE INVENTION

The coronary stent is the most important advance in interventional cardiology since the introduction of balloon angioplasty. In fact, percutaneous coronary revascularization now involves the use of a stent in about 70% of cases. Compared to balloon-based percutaneously transluminal coronary angioplasty (PTCA), the use of the coronary stent has reduced the rate of restenosis (narrowing of an artery that was previously opened by a cardiac procedure). However, the rate of restenosis is still too high, and in particular, in-stent restenosis (ISR; narrowing inside the stent) has become a significant problem for stent use.

A temporary inflammatory response with subsequent release of chemotactic and growth factors plays an important role in the genesis of restenosis. A stent, as a foreign body in the vessel, can induce a prolonged and serious inflammatory response in the vessel after its implantation. Patients with allergic patch-test reactions to nickel and molybdenum, which are components of many stents, have a higher frequency of ISR than patients without these reactions. Inflammatory cells—such as leukocytes, macrophages, and T lymphocytes—usually aggregate adjacent to stent struts. These inflammatory responses can potentiate the proliferation and extracellular matrix expression of smooth muscle cells and fibroblast cells (1). In addition, these inflammatory cells themselves can be integrated into the neointima, which is the main component in the narrowing of the lumenal cross-sectional area of the stented vessel (2).

Whatever the mechanism, ISR can lead to additional downstream complications. For example, thrombosis (formation of an obstructing clot inside a blood vessel) is another complication related to stent implantation. Thrombosis is a major complication in stent use, not only because of its frequency, but also because of its relation with serious outcomes, such as myocardial infarction. The rate of subacute thrombosis has decreased with improvements in stent design, deployment and anti-thrombotic therapy, but thrombosis remains a problem. Conventional stents reported to provide low rates of short-term restenosis were later found to have been subject to late in-stent thrombosis. Efforts to expand the potential clinical applications of vascular stents have included strategies to further reduce the thrombogenicity of metallic stents and to inhibit intimal thickening within the stent to reduce the incidence of restenosis. As a result, there is an expanding list of materials (e.g., collagen, fibrin and various drugs) being used to coat metal stents in an attempt to reduce ISR and thrombogenicity. Further, investigators have shown that the systemic and specific inhibition of inflammatory cells can decrease the in-stent restenosis in an animal model (3).

Nevertheless, eliminating restenosis has been difficult to achieve. Current methods for the prevention of ISR involve eliminating patient-related factors (e.g., more careful selection of patients) and procedure-related factors. The latter include better implantation techniques and stent design, stent coating with drugs and non-pharmaceutical agents, radioactive stents, intraluminal radiotherapy, gene therapy, etc. Procedures also include medical therapy, balloon angioplasty, directional atherectomy, rotational atherectomy, laser angioplasty, etc. However, none of these treatments are able to simultaneously achieve optimal safety, ease of use and low rate of restenosis.

Antiproliferative drug-eluting stents appear to be a very intriguing new therapy. These stents have achieved very low restenosis rates (from 0 to 3.1%) in limited populations over short terms (from 6 to 9 months after the implantation). However, there is a concern that this success comes at the cost of late in-stent thrombosis, late ISR, and a relatively high rate of myocardial infarction, as has occurred with β-brachytherapy and paclitaxel-eluting stent implantation. In fact, an exaggerated inflammatory response and exuberant neointimal reaction were found when polymers were impregnated on stents and implanted in a porcine model. Such polymers can be a particular problem in drug-eluting stents.

In view of the above, improved methods for reducing restenosis and its subsequent complications are required.

### SUMMARY OF THE INVENTION

In view of research showing that a foreign body introduced into the body of a rat, rabbit, or mouse for two weeks initiates an inflammatory response with a resultant capsule of granulation tissue surrounding the foreign body, the present inventors hypothesized that a medical device, such as a stent, implanted below abdominal skin (or other suitable location), would similarly lead to capsulation of the medical device in granulation tissue. In theory, the capsulated medical device could then be removed and therapeutically transplanted into a treatment site appropriate to the medical device. For example, a capsulated stent would be implanted in a blood vessel, which would treat the stent as endogenous tissue due to the covering of immuno-compatible material. As a result, the inflammatory response would decrease and both restenosis and thrombosis would also decrease. In contrast, if neointimal growth could be increased, thus thickening the vessel wall, it may prove beneficial in the treatment of aneurysms (a sac-like protrusion from a blood vessel or the heart, resulting from a weakening of the vessel wall or heart muscle).

In accord with the concepts disclosed herein, it is expected that the long term benefit of the disclosed capsulated medical device (e.g., a capsulated stent) will exceed that of conventional medical devices (e.g., conventional bare stents or drug-eluting stents) intended for the same end-use therapeutic application.

In accord with one aspect of the present concepts, a medical device is provided which includes an implantable medical device that is at least substantially covered in a granulation tissue. The granulation tissue is substantially immunocompatible with an immune system of a patient into which the implantable medical device is to be implanted for a therapeutic purpose.

In yet another aspect, a stent is provided which includes a granulation tissue covering which is substantially immunocompatible with an immune system of a patient into which the stent is to be implanted for a therapeutic purpose.

In accord with another aspect of the present concepts, a treatment method is provided which includes the acts of subcutaneously implanting a medical device into a patient and incubating the medical device for a period sufficient to allow the medical device to be at least substantially encapsulated by granulation tissue. Subsequently, the method includes the acts of removing the capsulated medical device from the patient and therapeutically implanting the capsulated medical device into the patient.

In accord with yet another aspect of the present concepts, a method of producing an implantable medical device for a subsequent therapeutic treatment of a patient is provided which includes the act of incubating a medical device for a period sufficient to allow the medical device to be at least partially encapsulated by granulation tissue. Further acts consistent with this method include subcutaneously implanting the medical device into a host. The host may be the patient designated for therapeutic treatment by the medical device.

In still another aspect of the present concepts, a vascular stent is substantially encapsulated (i.e., enclosed within or surrounded by) in granulation or granuloma tissue and then treated with an agent, such as an anti-inflammatory, or chemotherapeutic drug.

In yet another aspect of the present concepts, a capsulated stent is used without an anti-inflammatory drug. Implantation of the capsulated stent sans anti-inflammatory agent may be, for example, useful to increase neointimal growth as noted above.

The above summary of the present concepts is not intended to represent each embodiment, or every aspect, of the present concepts, which are set forth by way of example in the accompanying detailed description and figures and which are defined by the claims.

### BRIEF DESCRIPTION OF THE DRAWING

FIG. 1 is a representation of a capsulated stent with an inner tube or sleeve.

### DETAILED DESCRIPTION OF THE INVENTION

“Capsulated stent,” as used herein, comprises a stent that is at least substantially covered with a layer of granulation/granuloma tissue and includes, but is not limited to, a stent that is completely covered with a layer of granulation/granuloma tissue. The exact cellular and matrix composition of this “capsule” has not been completely characterized, but it is generally granuloma in origin and is generally host-compatible. “Encapsulating” refers to the process of covering or essentially enclosing the device with granulation tissue.

The terms “host-compatible” and “immunocompatable,” as used herein, mean the capsulated device elicits significantly less (at least 50% less) immune response than the device alone.

In a study of the above-noted hypothesis, as described herein, stents were pre-implanted subcutaneously in rabbit abdomen for encapsulation and were then subsequently implanted into endothelial cell-denuded vessels. The local inflammatory response and the ratio of neointima-to-media diameter in these vessels were measured. The effect of the granulation capsule was determined to be beneficial when the capsulated stent was combined with mitomycin C. Results of this studies, to date, have demonstrated that: 1) granulation tissue can cover the new stents on subcutaneous implantation; 2) capsules are strong enough to withstand the pressures of both implantation and arterial use; 3) capsulated stents treated with mitomycin C decrease the thickness of any resulting neointima in the vessel, as compared with capsulated stents lacking drug treatment; and 4) capsulated stents alone increase the thickness of resulting neointima, as compared with bare stents.

By implication, capsulated stents coated with inflammation reducing drugs will have a decreased incidence of complications, including restenosis and thrombosis, and may be advantageously employed in-lieu of conventional stents. In contrast, capsulated stents used without an anti-inflammatory drug result in increased neointima, and thus may be advantageously employed in-lieu of conventional stents whenever it is desired to increase vessel wall thickness or strength.

Inflammation inhibiting drugs include steroidal and non-steroidal anti-inflammatory drugs (NSAIDs), such as COX2 or ERK inhibitors, and the like. Chemotherapeutic drugs are also included as “anti-inflammatory” agents because they have been shown to inhibit inflammation by inhibiting the growth of inflammation cells. Such drugs include, but are not limited to, sirolimus or rapamycin, paclitaxel, Batimastat, and Actinomycin-D. One preferred DNA synthesis inhibiting drug is mitomycin C. The stents may also be treated with other beneficial drugs, such as antibiotics, anti-platelet drugs, and the like. Interestingly, the inventors' experiments have shown that the drug was retained even when the stent was washed before use, suggesting that the drug penetrated and was retained by the granulation tissue, providing a subsequent slow release. Thus, a drug-bearing granulation tissue provides, in accord with the present concepts, an alternative to the use of polymers for preparing an implantable drug delivery device (e.g., a drug-eluting stent).

In one aspect of the present concepts, a method of preparing a host-compatible capsulated stent includes subcutaneous implantation of the stent into the patient for a period sufficient to allow encapsulation. The stent may also be advantageously covered (e.g., in vitro) by incubation with cells, proteins and growth factors appropriate to a desired effect. This aspect of the method permits introduction of cells specially modified to address a particular need, such as by providing the fibrin gene to treat Marfan's syndrome, providing anti-coagulation proteins, or the like.

Generally, the above method relates not only to a method of preparing a host-compatible capsulated stent, but to a method of producing an implantable medical device for a subsequent therapeutic treatment of a patient including the step of incubating a medical device for a period sufficient to allow the medical device to be at least partially encapsulated by granulation tissue. The encapsulation is preferably achieved by subcutaneously implanting the medical device into a host, which host is preferably the patient designated for therapeutic treatment by the implantable medical device. The host may also include a mammalian host such as, but not limited to, a porcine or bovine host. Although not yet commercially realized, the potential exists for utilization of universal-host animals for xeno-transplantation purposes in accord with the present concepts to facilitate or complete the encapsulation process to produce a capsulated medical device in accord with the present concepts. This would eliminate the need for the patient who is to receive the implantable medical device for a subsequent therapeutic treatment to subcutaneously carry the medical device prior to therapeutic implantation (e.g., vascular implantation for a stent). Where the patient is both used to form in-situ a capsulated implantable medical device and to receive such device in a therapeutic end-use thereof, the above method further comprises removing the implantable medical device from the patient and, following an optional treatment thereof with a drug, therapeutically implanting the capsulated medical device into the patient.

As noted above, the capsulated implantable medical device (e.g., a stent) may be advantageously treated with one or more drugs prior to therapeutic implantation into the patient to provide a desired local or systemic effect to the patient. The granulation tissue has the effect of retaining the drug, slowly releasing it in situ. Thus, the invention provides a novel drug-eluting implantable medical device which comprises, in one aspect, a stent, but may comprise any capsulated implantable medical device treated to form a drug-eluting version thereof. Further, because artificial polymers are not used to provide a drug reservoir, the potential for reactions against the polymer are eliminated. The capsulated implantable medical device may also be advantageously treated with one or more drugs prior to subcutaneous implantation into the patient (or other host) to provide a desired local or systemic effect to the patient and/or to provide a desired characteristic to the encapsulation of the implantable medical device (e.g., a drug selectively enhancing or retarding the formation of granulation/granuloma tissue).

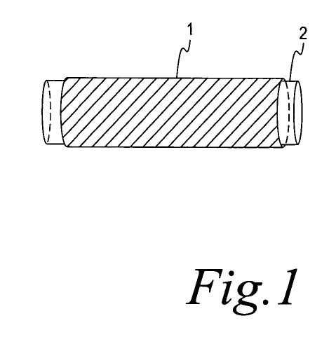

One representation of an exemplary configuration in accord with the present concepts is shown in FIG. 1. According to this illustration, an optional tube, sheath or sleeve 2 (hereinafter “tube”) is disposed inside of and preferably spaced apart from stent 1 by a predetermined spacing prior to subcutaneous implantation of the stent to limit or prevent the in-growth or overgrowth of granulation tissue, which can itself block the stent. Following formation of the granulation tissue on surfaces of the stent 1, as generally represented by the hatched lines in FIG. 1, the tube 2 is removed prior to subsequent implantation in a vessel.

Although depicted as a cylinder, tube 2 may optionally assume other forms (e.g., tapered) to achieve the end of selectively limiting or preventing the in-growth or overgrowth of granulation tissue within the stent. As shown by experiment, the presence of tube 2 within the stent 1 during the encapsulation process facilitates the formation of a substantially smooth or smooth surface on the interior of the stent. Alternatively, it may also be possible to closely calibrate the requisite granuloma tissue growth period, thereby eliminating the need for tube 2.

Material selection for the tube 2 can vary, depending on the therapeutic use, and may comprise any suitable medical-grade material including, for example, silastic, plastic, Teflon™, medical-grade stainless steel, and medical-grade titanium alloy. Silastic is one currently preferred material. In some aspects of the present concepts, it is preferred that the material of tube 2 comprises a material that is essentially inert in the body so as to prevent any growth on or reaction to the tube. In the experiments conducted by the inventors, tube 2 was removed prior to implantation of the capsulated stent into the blood vessel of the test subject. Likewise, tube 2 would be removed prior to a therapeutic use of the stent (i.e., implantion of the stent into a patient's blood vessel).

In another aspect, an exterior tube may optionally be disposed on an exterior of the stent 1, which may be used in isolation with the stent, in combination with the stent and exterior tube, or in combination with a drug or treatment on an inner surface of the stent to inhibit or promote formation of granulation tissue. Openings, spaces, channels or gaps may be optionally provided between the tube(s) and the stent. Spacers (not shown) may also be used to space apart the tube(s) from the stent. These physical barriers and/or drugs or treatments may advantageously permit formation of the granulatien tissue into a preferred geometry or bias the formation of the granulation tissue toward a preferred geometry. For example, a combination of an inner tube 2 and an outer tube with a stent 1 disposed therebetween may be useful in the formation of a capsulated stent having cylindrical shape of a substantially predetermined thickness. This concept may likewise be extended to other types of implantable medical devices.

In one aspect of the present concepts, the capsulated implantable medical device may be treated with an anti-inflammatory agent. In the example of a capsulated stent, such stent may be treated with mitomycin C (a cell cycle inhibitor), which has been shown to reduce neointimal formation. Therefore, capsulated stents in accord with the present concepts can be treated to decrease restenosis and its attendant complications. The stent, or other implantable medical device, may comprise any metal or polymer, provided the material is suitably biocompatible and has the requisite structural characteristics for its particular application.

The invention is exemplified in the attached examples, but has broader application than specifically exemplified herein.

### EXAMPLE 1

### Methods

A representation of a capsulated stent is shown in FIG. 1. According to this embodiment, tube 2 is disposed inside of stent 1 prior to subcutaneous implantation of the stent to limit or prevent the in-growth or overgrowth of granulation tissue, which can itself block the stent.

New Zealand White Rabbits, each weighing 3 to 4 kg, were used for these experiments. Animals were housed individually in steel mesh cages and fed rabbit chow and water. All procedures were performed under general anesthesia induced by intramuscular injection of 35 mg/kg IM ketamine (AVECO CO.™) and 0.2 mg/Kg acepromazine after pre-medication with 10 mg/kg IM xylazine (MILES, INC.™).

Stents were transplanted subcutaneously over the abdomen, or other suitable location, and removed after encapsulation, typically after two weeks. The inner tube was removed, and the capsulated stent was treated with the appropriate drug (such as an antibiotic, immunosuppressant, anti-inflammatory, or the like) and then mounted over a balloon for delivery to the blood vessels.

Capsulated stents at days 3, 7, and 14 (n=5 to 8 rabbits in each group, a total of 15 to 24; one stent per rabbit; the control was a bare stent) were transplanted into endothelial cell-denuded external iliac arteries (EIA) in the same rabbits after treatment with 100 μg/ml mitomycin C for 1 hour at 37° C. in CO₂ incubator under the same general anesthesia as described above. To reduce the significant incidence of early occlusive thrombosis, aspirin (SIGMA CHEMICAL CO™, 0.07 mg/ml) was added to the drinking water 1 day before surgery to achieve an approximate dose of 5 mg/kg/day.

A 5F introducer sheath was positioned in the femoral artery under surgical exposure, after which nitroglycerin 0.25 mg and heparin 1000 USP units were administered intra-arterially. All catheters were subsequently introduced through this sheath and advanced to the EIA via a 0.014-inch guidewire. Arterial injury was produced using a 3F Fogarty balloon catheter (BAXTER EDWARDS™) to denude the endothelial cells. Stent implantation was performed by introducing a 15 mm long Palmaz-Schatz coronary stent (JOHNSON & JOHNSON INTERVENTIONAL SYSTEMS™) over an 3F angioplasty balloon catheter (SCI-MED™). The stent was apposed to the vessel wall by high-pressure balloon inflation (10 atm inflation for 15 seconds) to achieve a 1.1 to 1.2:1.0 stent-to-artery ratio.

External iliac arteries (EIA) were harvested at days 14 and 28 (one stent per rabbit) and observed. Student's T-tests were used to determine whether there was an increase in 1) mean neointimal area; 2) ratio of neointimal diameter to media diameter between the group of rabbits implanted with the granulation tissue-covered stents and the control group; and 3) average of densities of monocytes/macrophages between the group of rabbits implanted with granulation tissue-covered stents and control group. An associated p-value of 0.05 was considered significant.

Additional experiments will further study the inflammatory response: Inflammatory responses will be measured by immunohistochemistry with antibodies to macrophages (RAM 11, DAKO CO.™) and neutrophils (monoclonal mouse RPN 3/57 IgG, SEROTEC, INC.™). Capsulated stents will be explanted from the rabbits at days 3, 7 and 14 after subcutaneous implantation. The granulation tissues will be fixed for 15 minutes in 4% paraformaldehyde fixative. Subsequently, tissues will be cleared and embedded with paraffin (melting point 58-60° C.) a 60° C. for 2 hours in a vacuum evaporating embedder. Tissues will be sectioned, deparaffinized, treated for 5 minutes with 3% hydrogen peroxide and blocked before incubation with the primary antibody and then with a biotinylated species-specific secondary antibody (VECTOR LABORATORIES INC.™). Cells will be “stained” by avidin-biotin peroxidase or avidin-biotin-alkaline phosphatase (VECTOR LABORATORIES INC.™). In these sections, overall tissue cell density will be calculated by dividing the number of nuclei by the granulation area around the stents. The number of immunologically identified monocytes/macrophages will be counted and the densities of these cell types calculated. Since RPN 3/57 IgG also identifies rabbit thymocytes, identification of cells as neutrophils will be further confirmed by examining serial sections for the characteristic morphology of cells under Verhoeff's stain (multilobulated nuclei and granulocytic cytoplasm).

### EXAMPLE 2

### Encapsulation of Stent

To test whether the stent could be subcutaneously encapsulated, and to confirm that the capsule was strong enough to resist the pressure used in implantation, stents were implanted and removed after a period of granulation and the stents were subjected to pressure, as follows:

Stents were implanted subcutaneously over the rabbit abdomen and removed after 14 days and observed. The stents were adequately encapsulated by granulation tissue. After being treated with 200 μg/ml mitomycin C and washed in saline for 30 minutes, the granuloma-capsulated stents were dilated by balloons using a pressure of 10 atm outside the vessels and 8 atm inside the iliac artery of the rabbit. Stents were then observed and the granulation capsules (inside and outside) were completely intact without any visible fractures. Furthermore, the capsules were still completely intact 30 hours after stent implantation into the iliac artery.

This work demonstrated that the granulation capsules were intact both inside and outside of the vessels after dilation with pressures from 8 to 10 atm. Several investigators have demonstrated that a foreign body introduced into the body of a rat, rabbit, or mouse for two weeks initiates an inflammatory response with a resultant capsule of granulation tissue (10-12). These granulation capsules are so strong that they could be used as arteries to resist normal artery pressure (10). Moreover, the granulation capsule over the stents can be molecularly engineered using different genes for the treatment of various arterial aneurysms. For instance, the granulation capsule can be modified with the fibrin gene to treat Marfan's syndrome, in which the lack of fibrin in the vessels often causes vascular aneurysm.

### EXAMPLE 3

### Neointimal Formation

In order to determine the effect of a capsulated stent in situ, neointimal formation was studied as follows:

As in examples 1 and 2, stents were implanted subcutaneously over the rabbit abdomen for 14 days. Both capsulated and bare stents were then implanted in rabbit iliac arteries and, at the appropriate time (4 weeks), the arteries were excised and analyzed.

The vessels with capsulated stents treated by saline had significantly more neointimal area than did the vessels with bare stents (3.58±0.12 vs. 1.15±0.10 mm², p<0.05). The average injury scores between these two groups showed no significant differences (1.38±0.31 vs. 1.51±0.32, P>0.05).

The granulation capsulated stents treated with mitomycin C had significantly less neointimal area than the bare stents had (0.27±0.03 vs. 1.15±0.08 mm², P<0.05), and their injury scores showed no significant differences (1.46±0.18 vs. 1.51±0.32, P>0.05).

The inhibition of neointimal formation in the vessel is presently believed to arise for two reasons. First, the body treats the capsulated stents as self-tissue and does not initiate the inflammatory reaction, which has been proven to be a major contributor to in-stent restenosis (1-3). Secondly, it is possible that the mitomycin C penetrated the granulation tissue and was not totally washed away by the saline wash, but was slowly released after implantation. Mitomycin C, an alkylating agent, can inhibit local inflammatory cells from dividing (4-5). Therefore, these stents worked essentially as a “drug eluting stent,” slowing down the action of any inflammatory response that may have been initiated.

The granulation capsulated stent, which is biocompatible and hemocompatible, has advantages over the drug eluting stents currently available in the market. For instance, available drug eluting stents have a non-erodable polymer matrix, which itself can cause an inflammatory response and neointimal formation in vessels (6-9). In addition, by prolonging the wash time or drug treatment time, the concentration of drug combined with granulation capsules can be controlled.

In accord with the above-disclosed examples and concepts, a method of producing an implantable medical device for a subsequent therapeutic treatment of a patient and a medical device and a treatment method relating thereto are disclosed. Variations on these themes are also considered to be embodied within the present concepts. For example, further to the above, the material used for the optional tube 2 and for the stent 1 can be tailored to suit the desired therapeutic use. Thus, for example, materials can be selected to inhibit stimulation of granulation tissue formation or to stimulate granulation tissue formation, to varying degrees. Tube 2 and/or stent 1 could also optionally be modified by chemical, physical, and biomedical methods, such as by coating stent 1 with protein, such as collagen, or other material to help cover the stent with granulation tissue. Chemicals, such as polyethylene glycol (PEG), can be used and the stents may even be pre-seeded with immuno-compatible cells, other cell-types, or drugs.

Similarly, depending on the therapeutic use, the capsulated medical device, stents in the above examples, can be treated with different drugs to either inhibit granulation tissue and new intimal formation, or to stimulate granulation and new intimal formation. Other drugs can be employed, such as antibiotics and immunosuppressants.

Further to the above-disclosure, each of the references listed below is incorporated by reference in its entirety.

1. Amano J, et al., Proliferation of smooth muscle cells in acute allograft vascular rejection, J. Thoracic Cardiovasc. Surg. 1997; 113(1):19-25.

2. Murakami T, Yamada N, Modification of macrophage function and effects on atherosclerosis, Current Opinion in Lipidology 1996; 7(5):320-323.

3. Grewe P H, et al., Acute and chronic tissue response to coronary stent implantation: Pathologic findings in human specimens, J. Am. Coll. Cardiol. 2000; 35:157-163.

4. Tang L L, et al., Genetically engineered biologically based hemostatic bioassay, Ann. Biomed. Engin. 2003; 31:159-162.

5. Philips F S, et al, Pharmacology of miomycin C. Toxocity and pathologic effects, Cancer Res. 1960; 20:134-1361.

6. Suzuki T, et al., Stent-based delivery of sirolimus reduces neointimal formation in a porcine coronary model, Circulation. 2001; 104(10): 1188-93.

7. van der Giessen W J, et al., Marked inflammatory sequelae to implantation of biodegradable and nonbiodegradable polymers in porcine coronary arteries, Circulation. 1996;94:1690-1697.

8. Shurmann K, et al., Biologic response to polymer-coated stents: In vitro analysis and results in an iliac artery sheep model, Radiology 2004; 230:151-162.

9. Smith T P, Why coat a stent with polymer? Radiology 2004; 230:1-2.

10. Campbell J H, et al., Blood vessels from bone marrow, Ann. New York Acad. Sci. 2000; 902:224-229.

11. Ryan G B, et al., Myofibroblasts in an avascular fibrous tissue, Labor. Invest. 1973; 29(2):197-206.

12. Mosse P R, et al., A comparison of the avascular capsule surrounding free floating intraperitoneal blood clots in mice and rabbits, Pathology 1985; 17(3):401-7.

While the present invention has been described with reference to one or more particular embodiments, those skilled in the art will recognize that many changes may be made thereto without departing from the spirit and scope of the present concepts. Each of these embodiments and obvious variations thereof is contemplated as falling within the spirit and scope of the claimed invention, which is set forth in the following claims.

1. A medical device, comprising:
an implantable medical device at least substantially covered in a granulation tissue,
wherein the granulation tissue is substantially immunocompatible with an immune system of a patient into which the implantable medical device is to be implanted for a therapeutic purpose.

2. The medical device according to claim 1, wherein the granulation tissue bears a drug for release into the patient.

3. The medical device according to claim 2, wherein the drug comprises at least one of an anti-coagulation agent, anti-platelet agent, antibiotic, and anti-inflammatory agent.

4. A stent, comprising:
a granulation tissue covering,
wherein the granulation tissue is substantially immunocompatible with an immune system of a patient into which the stent is to be implanted for a therapeutic purpose.

5. The stent according to claim 4, wherein the granulation tissue bears a drug.

6. The stent according to claim 5, wherein the drug comprises at least one of an anti-coagulation agent, anti-platelet agent, antibiotic, and anti-inflammatory agent.

7. The stent according to claim 4, wherein the granulation tissue covering covers substantially all of the stent.

8. The stent according to claim 4,
wherein the granulation tissue on an inner portion of the stent has a predetermined thickness.

9. The stent according to claim 8,
wherein said predetermined thickness substantially comprises a difference between an inner diameter of the stent and an outer diameter of a tube placed within said stent.

10. The stent according to claim 9,
wherein said tube comprises at least one of silastic, plastic, Teflon™, surgical stainless steel, and medical grade titanium alloy.

11. A treatment method, comprising the act of:
subcutaneously implanting a medical device into a patient;
incubating the medical device for a period sufficient to allow the medical device to be at least substantially encapsulated by granulation tissue;
removing the capsulated medical device from the patient; and
therapeutically implanting the capsulated medical device into the patient.

12. A treatment method according to claim 11, further comprising the act of:
treating the capsulated medical device with a drug prior to said therapeutically implanting act.

13. A treatment method according to claim 12,
wherein the drug comprises at least one of an anti-coagulation agent, anti-platelet agent, antibiotic, and anti-inflammatory agent.

14. A treatment method according to claim 11,
wherein said incubating act comprises incubating the medical device for a period sufficient to allow the medical device to be completely encapsulated by granulation tissue.

15. A treatment method according to claim 12,
wherein said act of treating comprises treating the capsulated medical device with a drug for a period sufficient to allow the drug to at least partially penetrate the granulation tissue so as to permit retention of a therapeutic amount of the drug by the granulation tissue.

16. A treatment method according to claim 11,
wherein said medical device is a stent.

17. A treatment method according to claim 11,
wherein said act of subcutaneously implanting comprises subcutaneously implanting a stent comprising an inner sleeve.

18. A treatment method according to claim 17, further comprising the act of:
removing the inner sleeve from the capsulated stent following said act of removing the capsulated medical device from the patient.

19. A method of producing an implantable medical device for a subsequent therapeutic treatment of a patient, comprising the act of:
incubating a medical device for a period sufficient to allow the medical device to be at least partially encapsulated by granulation tissue.

20. The method of producing an implantable medical device according to claim 19, further comprising, prior to the act of incubating:
subcutaneously implanting the medical device into a host.

21. The method of producing an implantable medical device according to claim 20,
wherein the host is the patient designated for therapeutic treatment by the implantable medical device.

22. The method of producing an implantable medical device according to claim 20,
wherein the host is not the patient designated for therapeutic treatment by the implantable medical device.

23. The method of producing an implantable medical device according to claim 21, further comprising the act of,
removing the at least partially encapsulated medical device from the patient.

24. The method of producing an implantable medical device according to claim 23, further comprising the act of:
treating the at least partially capsulated medical device with a drug prior to a therapeutic implantation thereof into the patient.

25. The method of producing an implantable medical device according to claim 24,
wherein said act of treating comprises treating the capsulated medical device with a drug for a period sufficient to allow the drug to at least partially penetrate the granulation tissue so as to permit retention of a therapeutic amount of the drug by the granulation tissue.

26. The method of producing an implantable medical device according to claim 24, wherein the medical device is a stent.
